(19) United States
(12) Patent Application Publication
(10) Pub. No.: US 2026/0325829 A1
(43) Pub. Date: Oct. 22, 2026

(54) INTRAVASCULAR BLOOD PUMP WITH A FILTER UPSTREAM OF A BLOOD PUMP INLET

(71) Applicant: ABIOMED, INC., Danvers, MA (US)

(72) Inventors: Cam LOUGHLIN, Danvers, MA (US); Jun DING, Danvers, MA (US); Jairo Leandro MOUX, Danvers, MA (US); James MELTON, Danvers, MA (US); Emily HEIL, Danvers, MA (US); Charles PERKINS, Danvers, MA (US)

(73) Assignee: ABIOMED, INC., Danvers, MA (US)

(21) Appl. No.: 19/450,430

(22) Filed: Jan. 15, 2026

Related U.S. Application Data
(60) Provisional application No. 63/746,706, filed on Jan. 17, 2025.

(51) Int. Cl. A61M 60/888; A61M 60/13; A61M 60/36

(52) U.S. Cl. CPC A61M 60/888; A61M 60/13; A61M 60/36

(57) ABSTRACT

An intravascular blood pump includes a pump section and a filter section. The pump has a blood flow inlet, a blood flow outlet, and an impeller that is configured to cause blood to flow into the blood flow inlet towards the blood flow outlet. The filter section is disposed upstream of the blood flow inlet. The filter section has a central body extending along a longitudinal axis and an expandable structure disposed over the central body. The expandable structure is configured to radially expand away from the central body in an expanded configuration and direct blood flow toward the central body in the expanded configuration.

### CROSS-REFERENCE TO RELATED APPLICATIONS

This application claims the benefit of priority under 35 U.S.C. § 119 to prior filed U.S. Provisional Patent Application No. 63/746,706, filed Jan. 17, 2025 (Attorney Docket No.: 266489.000006 (ABD0307USPSP1/D0307.US01)), the entire contents of which is hereby incorporated by reference as if set forth in full herein.

### FIELD

The present disclosure relates generally to medical devices, and in particular to catheter-based blood pumps having a filter upstream of an inlet to the blood pump.

### BACKGROUND

This disclosure relates to an intravascular blood pump for percutaneous insertion into a patient's blood vessel, to support a blood flow in a patient's blood vessel. This disclosure particularly relates to a right ventricular blood pump to support a blood flow from the vena cava through the right ventricle into the pulmonary artery.

Intravascular blood pumps are inserted into a patient's vessel such as the aorta or vena cava and through a cardiac valve by means of a catheter and can also be referred to as catheter pumps. A blood pump typically comprises a pump section with a blood flow inlet and a blood flow outlet. To cause a blood flow from the blood flow inlet to the blood flow outlet, typically an impeller or rotor is rotatably supported within the pump casing about an axis of rotation for conveying blood. The blood pump may be driven by a motor included in the blood pump adjacent to the pump section or may alternatively be driven by a motor outside the patient's body, in which case the motor is connected to the impeller by a flexible drive shaft extending through the catheter.

A right ventricular blood pump is inserted through the inferior or superior vena cava through the right ventricle of a patient's heart into the pulmonary artery by means of a catheter. Typically, the blood flow inlet of the blood pump is placed inside the right atrium, vena cava or right ventricle, while the pump section extends through the tricuspid valve, the right ventricle and the pulmonary valve into the pulmonary artery.

Any blood clots or thrombi that occur may be conveyed from the vena cava to the pulmonary artery, which, however, does not cause severe harm to the patient because the thrombi only end up in the pulmonary circulation. More importantly, blood clots tend to clog the blood pump and thus may cause failure of the blood pump, which should be avoided.

### SUMMARY

The present disclosure is directed to an intravascular blood pump that includes a pump section and a filter section. The pump has a blood flow inlet, a blood flow outlet, and an impeller that is configured to cause blood to flow into the blood flow inlet towards the blood flow outlet. The filter section is disposed upstream of the blood flow inlet. The filter section has a central body extending along a longitudinal axis and an expandable structure disposed over the central body. The expandable structure is configured to radially expand away from the central body in an expanded configuration and direct blood flow toward the central body in the expanded configuration.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1 is an illustration of an example intravascular pump and filter inserted into a heart according to aspects of the present disclosure.

FIG. 2 is an enlarged view of the filter and pump inlet according to aspects of the present disclosure.

FIG. 3 is an enlarged view of the filter and pump inlet with the filter disposed within a sheath.

FIG. 4 is a cross-sectional view, with parts broken away, of an introducer having an inner sheath and an outer sheath for receiving the pump assembly.

FIG. 5. is a cross-sectional view, with parts broken away, of the introducer with the pump assembly inserted therein with a portion of the filter being unsheathed.

FIG. 6A is a view of the pump assembly with the distal ends of the struts of the filter being shown unconstrained.

FIG. 6B is an enlarged view of the filter shown in FIG. 6A.

FIG. 7A is an enlarged view of an alternative filter.

FIG. 7B is a top view of the filter shown in FIG. 7A.

FIG. 7C is a perspective view of the filter shown in FIG. 7A.

FIG. 7D is a side view of the filter shown in FIG. 7A.

### DETAILED DESCRIPTION

The following detailed description should be read with reference to the drawings, in which like elements in different drawings are identically numbered. The drawings, which are not necessarily to scale, depict selected embodiments and are not intended to limit the scope of the disclosure. The detailed description illustrates by way of example, not by way of limitation, the principles of the disclosure. This description will clearly enable one skilled in the art to make and use the disclosure, and describes several embodiments, adaptations, variations, alternatives and uses of the disclosure, including what is presently believed to be the best mode of carrying out the disclosure.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

In addition, as used herein, the terms “patient,” “host,” “user,” and “subject” refer to any human or animal subject and are not intended to limit the systems or methods to human use, although use of the subject disclosure in a human patient represents a preferred embodiment. As well, the term “proximal” indicates a location closer to the operator whereas “distal” indicates a location further away to the operator or physician.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

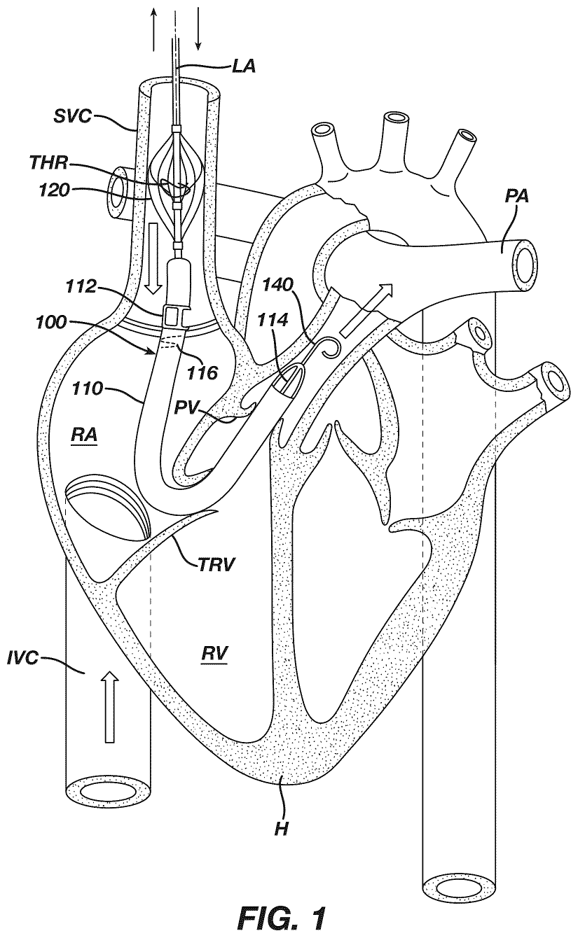

Referring now to FIGS. 1-3, an intravascular blood pump 100 includes a pump section 110, and a filter section 120. Pump section 110 comprises a blood flow inlet 112, a blood flow outlet 114, and an impeller 116 configured to cause blood to flow into the blood flow inlet 112 and towards the blood flow outlet 114. The pump section 110 further includes a motor 117 that is disposed in a motor housing 118. Motor 117 is configured to drive the impeller 116. Alternatively, the motor 117 may located outside the patient's body, in which case the motor is connected to the impeller 116 by a flexible drive shaft (not shown) extending through the catheter.

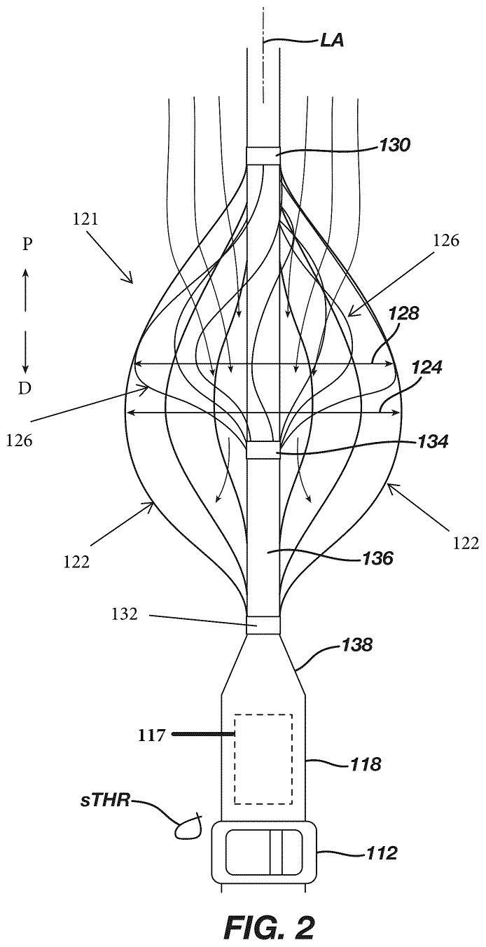

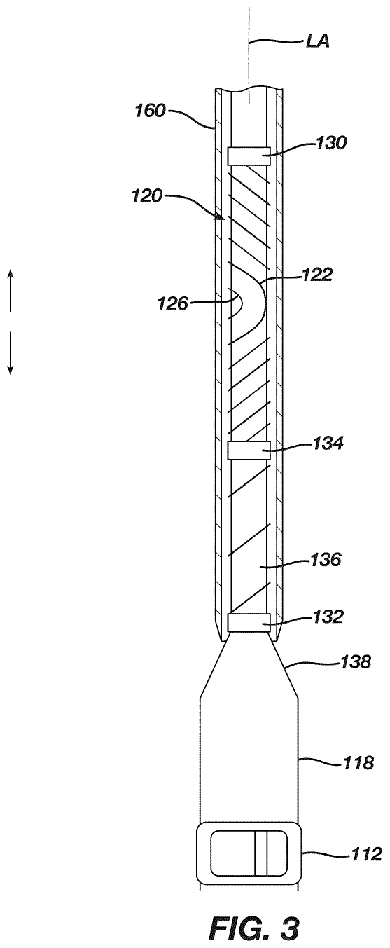

Filter section 120 is disposed upstream of the blood flow inlet 112. Filter section 120 includes a central body 136 extending along a longitudinal axis (LA) and an expandable structure 121 disposed over the central body 136. The expandable structure 121 is movable between a delivery configuration, an expanded configuration, and a removal configuration. In the expanded configuration, the expandable structure 121 is configured to radially expand away from the central body 136 and direct blood flow toward central body 136, as shown, for example, in FIG. 2. In the delivery configuration, the expandable structure 121 is radially constrained by a sheath 160, as shown, for example, in FIG. 3. The expandable structure 121 is configured to be deliverable via 23 Fr sheath. The removal configuration is similar to the delivery configuration shown in FIG. 3. The expandable structure 121 can be inserted and removed through the vascular access opening. The expandable structure 121 is configured to resiliently expand from the delivery configuration to the expanded configuration upon removal from sheath 160. The expandable structure 121 is configured to resiliently collapse from the expanded configuration to the removal configuration upon being slid back into sheath 160 before removal of the intravascular blood pump from vasculature. At least a portion of the expandable structure 121 spirals about the central body 136 in the delivery configuration. The removal configuration is distinct from the delivery configuration. In the delivery configuration, the expandable structure 121 comprises a plurality of struts each respectively comprising a first strut portion which spirals clockwise about a first portion the central body 136 and a second strut portion which spirals counterclockwise about a second portion of the central body 136. The expandable structure is collapsed within an insertion sheath in the delivery configuration. To remove the expandable structure 121, a secondary sheath can be advanced over the expandable structure to collapse the expandable structure 121 into the removal configuration before being removed through the delivery sheath 160.

As shown in FIG. 1, the intravascular blood pump 100 is configured to be positioned such that the filter section 120 is disposed in a superior vena cava (“SVC”) and the blood flow outlet 114 is disposed in a pulmonary artery (“PA”). Alternatively, the filter section 120 could be disposed in the inferior vena cava (“IVC”) and the blood flow outlet 114 could be disposed in the PA. In the expanded configuration, the expandable structure 121 is configured to abut walls of the SVC. In this position, the expandable structure 121 is configured to capture thrombus THR, as shown in FIG. 1. As shown in FIG. 2, the expandable structure 121 is configured to direct thrombus toward the central body 136 and retain the thrombus within the filter section 120. The filter section is configured to inhibit thrombus that is large enough to impede the impeller from encountering the blood flow inlet 112 due to blood flow caused by the impeller 116. The filter section is configured to allow small thrombus (“sTHR”) to encounter the blood flow inlet (112) such that the small thrombus is of a size such that the sTHR is capable of entering the blood flow inlet 112 and exiting the blood flow outlet 114 without impeding impeller 116.

The expandable structure 121 comprises a plurality of struts that are shaped to direct blood flow toward the central body 136 when in the expanded configuration. Every strut of the expandable structure 121 has two ends such that one of the two ends is upstream of the other of the two ends. As shown in FIG. 2, filter section 120 is disposed in a proximal direction in relation to pump section 110. The upstream direction and downstream direction are defined by a blood flow direction (BFD) across the filter section 120 and through the pump section 110. In one aspect of the disclosure, the struts can have a diameter of about 0.5 mm to about 1.0 mm.

The expandable structure 121 is comprised of a shape memory or superelastic material, or an alloy thereof, formed into a predetermined shape such that the expanded configuration is based at least in part on the predetermined shape. In one example, the shape memory material may be, for example, nitinol. The shape memory material could also be a flexible plastic like a thermoplastic polyurethane (TPU) or a poly(methyl methacrylate) (PMMA). The elongated catheter body is configured to be manipulated to position the pump section and the filter section within a heart, as shown in FIG. 1. Filter section 120 is disposed upstream of pump section 110. Filter section 120 comprises a central body 136 extending along a longitudinal axis (LA) and an expandable structure 221 disposed over the central body 136. The expandable structure 121 is configured to radially expand away from the central body 136 in an expanded configuration.

In accordance with one aspect of the disclosure, at least a portion of the plurality of struts spiral about the central body 136 in the expanded configuration. The expandable structure 121 comprises an outer strut assembly 122 and an inner strut assembly 126. The outer strut assembly 122 defines an inner volume in the expanded configuration and at least a majority of the inner strut assembly 126 is disposed within the inner volume, as shown in FIG. 2. The outer strut assembly 122 forms a lemon shape in the expanded configuration. The inner strut assembly 126 forms an onion shape in the expanded configuration. At least a portion of each strut of the inner strut assembly 126 spirals about the central body 136 in the expanded configuration. In the expanded configuration, an outer diameter 124 of the outer strut assembly 122 is greater than an outer diameter 128 of the inner strut assembly 126. Also, in the expanded configuration, the outer diameter 124 of the outer strut assembly 122 is greater than an average inner diameter of a human superior vena cava.

Filter section 120 further comprises an upstream anchor 130, a downstream anchor 132, and a central anchor 134 each being coupled to the central body 136 upstream of the pump section 110. Upstream anchor 130 is coupled to central body 136 upstream of the downstream anchor 132. Central anchor 134 is coupled to central body 136 between the downstream anchor 132 and the upstream anchor 130. An upstream end of the outer strut assembly 122 is coupled to the central body 136 by the upstream anchor 130. A downstream end of the outer strut assembly 122 is coupled to the central body 136 by the downstream anchor 132. An upstream end of the inner strut assembly 126 is coupled to the central body 136 by the upstream anchor 130. A downstream end of the inner strut assembly 126 is coupled to the central body 136 by the central anchor 134.

One anchor of the upstream anchor 130 and the downstream anchor 132 is configured to slide along the central body 136 in the direction of the longitudinal axis (LA). The other anchor of the upstream anchor 130 and the downstream anchor 132 is affixed to the central body 136 and stationary with respect to the longitudinal axis. In one aspect of the present disclosure, anchors 132 and 134 are fixed to central body 136, and anchor 130 is slidably received on central body 136. The expandable structure 121 can then move from the delivery configuration, shown in FIG. 3, to the expanded configuration, shown in FIG. 2, upon removal from sheath 160. Upon removal from sheath 160, expandable structure will self-expand, causing upstream anchor 130 to slide in the downstream direction towards central anchor 134 and downstream anchor 132 to the position illustrated in, for example, FIG. 2. The expandable structure 121 is configured to resiliently collapse from the expanded configuration to the removal configuration upon being slid back into sheath 160 before removal of the intravascular blood pump from the vasculature. Upon being slid back into sheath 160, expandable structure 121 will gradually collapse, causing upstream anchor 130 to slide in the upstream direction about central body 136 and away from central anchor 134 and downstream anchor 132 to the position illustrated in, for example, FIG. 3. In accordance with another aspect of the present disclosure, anchor 130 can be rigidly fixed to the catheter (136) and the expandable structure 121 is shaped in a way that allows it to radially collapse without requiring axial lengthening of the structure.

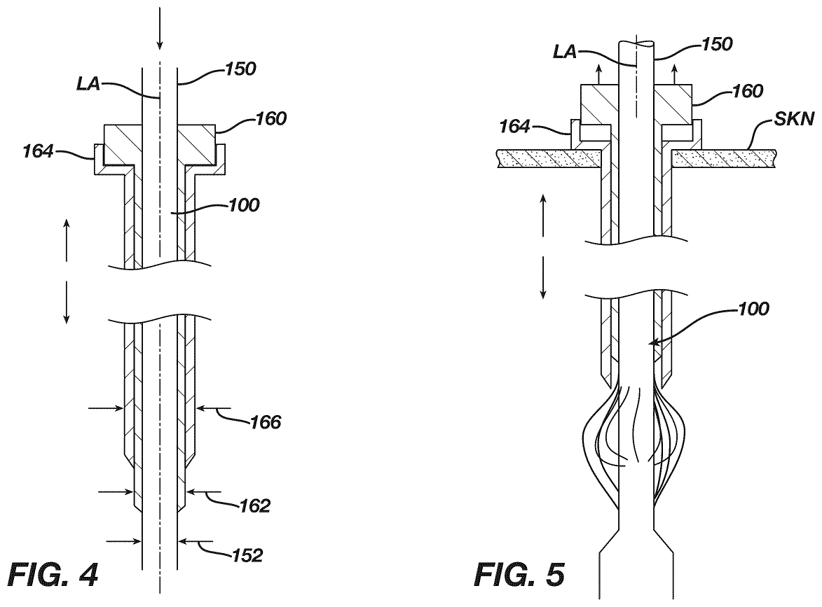

An outer sheath 164 is shown in FIGS. 4-5 penetrating through the skin (“SKN”) of a patient. Inner sheath 160 is configured to be delivered through the outer sheath 164. In accordance with one aspect of the present disclosure, inner sheath 160 is a 23 French size having an inner diameter of about 8.5 cm and an outer diameter of about 7.7 cm. Inner sheath 160 is configured to only partially collapse the expandable structure 121 from the expanded configuration. Inner sheath 160 is configured to collapse the expandable structure 121 from the expanded configuration to a diameter less than an inner diameter of the inner sheath 160, and wherein the expandable structure 121 is configured to further collapse for removal through the outer sheath 164 upon encountering a distal end of the outer sheath 164.

As shown in FIG. 2, in the expanded configuration, the expandable structure 122 is configured to direct blood flow toward the central anchor 134. As such, any thrombus THR or other potential obstruction in the blood can be captured in the expandable structure as shown, for example, in FIGS. 1 and 2.

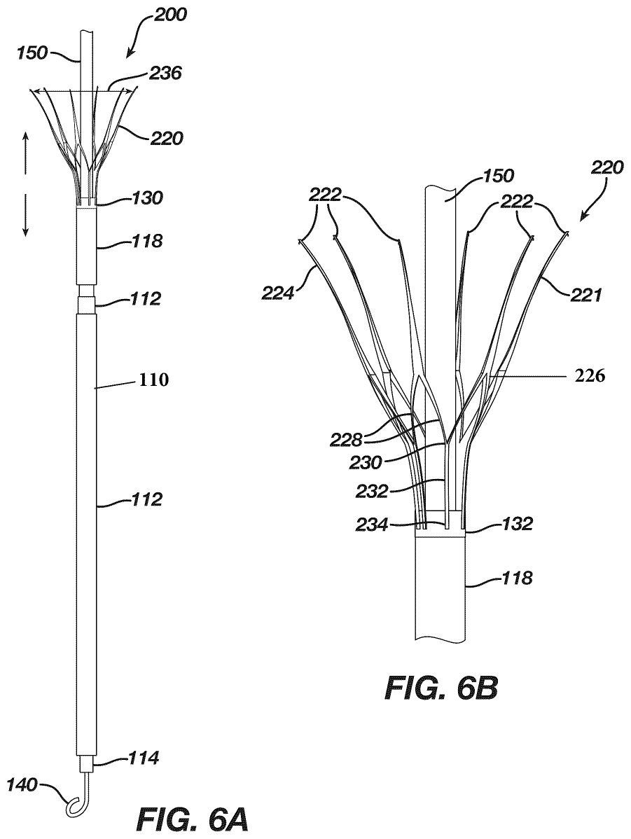

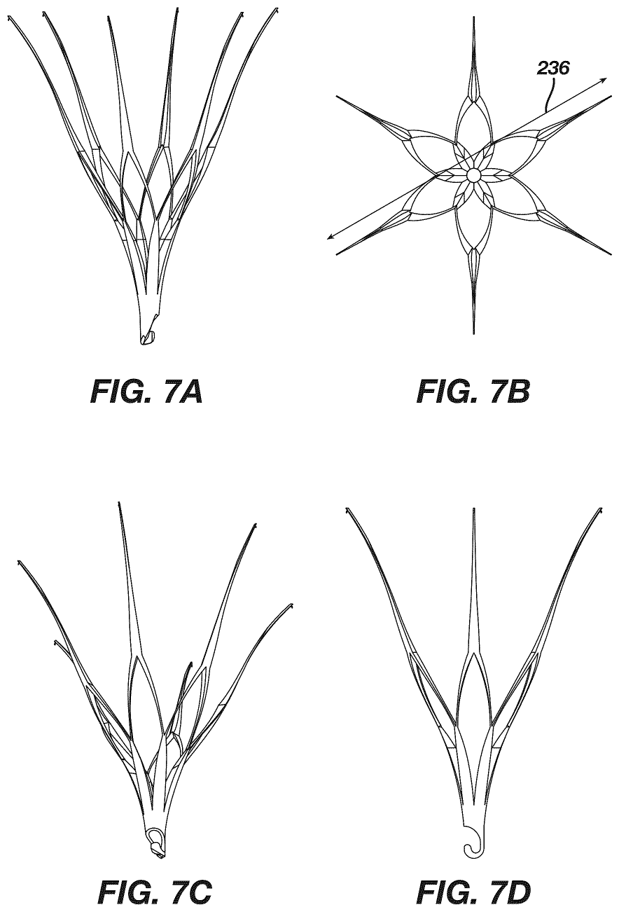

Referring now to FIGS. 6A-7D, another example of a filter section 220 to be used with an intravascular blood pump 200 is illustrated. Blood pump 200 includes a pump section 110 comprising a blood flow inlet 112, a blood flow outlet 114, and an impeller 116 configured to cause blood to flow into the blood flow inlet 112 and towards the blood flow outlet 114. A filter section 220 is disposed upstream of the pump section 110. Filter section 220 comprising a central body 136, 150 extending along a longitudinal axis LA and an expandable structure 221 disposed over or radially about the central body 136, 150. The expandable structure 221 is configured to radially expand away from the central body 150 in an expanded configuration, as shown in FIGS. 6A and 6B.

The expandable structure 221 comprises a plurality of struts arranged in a double wishbone configuration. The struts have an upstream end 222 and a downstream end 234. The expandable structure also has a plurality of upstream strut extensions 224 and a plurality of downstream strut extensions 232. The upstream strut extensions extend downstream respectively from each of the upstream strut ends 222. The downstream strut extensions 232 extending upstream respectively from each of the downstream strut ends 234. A plurality of wishbone struts 228 join the upstream strut extensions 224 to the downstream strut extensions 232 such that the plurality of wishbone struts 228 extend from the plurality of upstream strut extensions at upstream wishbone splits 226 and the plurality of wishbone struts 228 extend from the plurality of downstream strut extensions at downstream wishbone splits 230.

An anchor 132 is fixedly connected to central body 150. The strut ends 234 are each affixed to the central body 136 by anchor 132. The expandable structure 221 extends upstream of anchor 132. As shown, the upstream strut ends 222 are free ends. In addition, every strut of the expandable structure 221 is non-orthogonal with respect to the longitudinal axis LA. Just like blood pump 100, intravascular blood pump 200 is configured to be positioned such that the filter section 220 is disposed in a superior vena cava and the blood flow outlet 114 is disposed in a pulmonary artery. In this position, the expandable structure 221 is configured to abut walls of the superior vena cava in the expanded configuration. The free ends of the upstream struts are compliant enough so as not damage the SVC. The expandable structure 221 is made of a shape memory material, such as, for example, nitinol. The expandable structure 221 is formed into a predetermined shape such that the expanded configuration is based at least in part on the predetermined shape. In the expanded configuration, an outer diameter 236 of the expandable structure 221 is greater than an average inner diameter of a human superior vena cava. The filter shown in FIGS. 6A-B and 7A-D does not have any struts that extend around the entire circumference of the filter. Stated in other words, the expandable structure 221 lacks axial struts.

Like the example of the present disclosure illustrated in FIGS. 1-5, in the delivery configuration, the expandable structure 221 is radially constrained by a sheath 160. The expandable structure 221 is configured to resiliently expand from the delivery configuration to the expanded configuration upon removal from sheath 160. The expandable structure 221 is configured to resiliently collapse from the expanded configuration to the removal configuration upon being slid back into sheath 160 before removal of the intravascular blood pump from the vasculature.

The following clauses list non-limiting embodiments of the disclosure:

Clause 1. An intravascular blood pump (100, 200) comprising:
a pump section (110) comprising a blood flow inlet (112), a blood flow outlet (114), and an impeller (116) configured to cause blood to flow into the blood flow inlet (112) and towards the blood flow outlet (114); and
a filter section (120, 220) disposed upstream of the blood flow inlet (112), the filter section (120, 220) comprising a central body (136) extending along a longitudinal axis (LA) and an expandable structure (121, 221) disposed over the central body (136), the expandable structure (121, 221) being configured to radially expand away from the central body (136) in an expanded configuration and direct blood flow toward the central body (136) in the expanded configuration.

Clause 2. The intravascular blood pump (100, 200) of clause 1,
wherein the intravascular blood pump (100) is configured to be positioned such that the filter section (120, 220) is disposed in a superior vena cava (SVC) and the blood flow outlet (114) is disposed in a pulmonary artery (PA), and
wherein the expandable structure (121, 221) is configured to abut walls of the superior vena cava (SVC) in the expanded configuration.

Clause 3. The intravascular blood pump (100, 200) of clause 1 or 2, wherein the expandable structure (121, 221) is configured to capture thrombus (THR, FIG. 1).

Clause 4. The intravascular blood pump (100, 200) of any one of clauses 1-3, wherein the expandable structure (121, 221) is configured to direct thrombus (THR) toward the central body (136) and retain the thrombus within the filter section (120, 220).

Clause 5. The intravascular blood pump (100, 200) of any one of clauses 1-4, wherein the filter section is configured to inhibit thrombus (THR) large enough to impede the impeller from encountering the blood flow inlet (112) due to blood flow caused by the impeller (116).

Clause 6. The intravascular blood pump (100, 200) of any one of clauses 1-5, wherein the filter section is configured to allow small thrombus (sTHR, FIG. 2) to encounter the blood flow inlet (112) such that the small thrombus (sTHR) is of a size capable of entering the blood flow inlet (112) and exiting the blood flow outlet (114).

Clause 7. The intravascular blood pump (100, 200) of any one of clauses 1-6, wherein the expandable structure (121, 221) comprises a plurality of struts shaped to direct blood flow toward the central body (136) in the expanded configuration.

Clause 8. The intravascular blood pump (100, 200) of clause 7, wherein every strut of the expandable structure (121, 221) comprises two ends such that one of the two ends is upstream of the other of the two ends.

Clause 9. The intravascular blood pump (100, 200) of any one of clauses 1-8, wherein the expandable structure (121, 221) comprises a delivery configuration in which the expandable structure is radially constrained by a sheath (160).

Clause 10. The intravascular blood pump (100, 200) of any one of clauses 1-9, wherein the expandable structure (121, 221) comprises a delivery configuration and a removal configuration such that the expandable structure (121) is configured to resiliently expand from the delivery configuration to the expanded configuration upon removal from a sheath (160) and be collapsed by the sheath (160) to the removal configuration.

Clause 11. The intravascular blood pump (100, 200) of any one of clauses 1-10, wherein the filter section (120, 220) is disposed in a proximal direction in relation to the pump section (110).

Clause 12. The intravascular blood pump (100, 200) of any one of clauses 1-11, wherein an upstream direction and a downstream direction are defined by a blood flow direction (BFD) across the filter section (120, 220) and through the pump section (110).

Clause 13. The intravascular blood pump (100, 200) of any one of clauses 1-12, wherein the expandable structure (121, 221) comprises a memory shape material formed into a predetermined shape such that the expanded configuration is based at least in part on the predetermined shape.

Clause 14. The intravascular blood pump (100, 200) of any one of clauses 1-13, further comprising:
an elongated catheter body configured to be manipulated to position the pump section and the filter section within a heart.

Clause 15. The intravascular blood pump (100, 200) of any one of clauses 1-14, wherein the pump section (110) further comprises a motor (117) disposed in a motor housing (118) and configured to drive the impeller (116).

Clause 16. The intravascular blood pump (100) of any one of clauses 1-15, wherein at least a portion of the plurality of struts spiral about the central body (136) in the expanded configuration.

Clause 17. The intravascular blood pump (100) of any one of clauses 1-16, wherein the expandable structure (121) comprises an outer strut assembly (122) and an inner strut assembly (126) such that the outer strut assembly (122) defines an inner volume in the expanded configuration and at least a majority of the inner strut assembly (126) is disposed within the inner volume.

Clause 18. The intravascular blood pump (100) of clause 17, wherein the outer strut assembly (122) forms a lemon shape in the expanded configuration.

Clause 19. The intravascular blood pump (100) of clause 17 or 18, wherein the inner strut assembly (126) forms an onion shape in the expanded configuration.

Clause 20. The intravascular blood pump (100) of any one of clauses 17-19, wherein at least a portion of each strut of the inner strut assembly (126) spirals about the central body (136) in the expanded configuration.

Clause 21. The intravascular blood pump (100) of any one of clauses 17-20, wherein, in the expanded configuration, an outer diameter (124) of the outer strut assembly (122) is greater than an outer diameter (128) of the inner strut assembly (126).

Clause 22. The intravascular blood pump (100) of any one of clauses 17-21, wherein, in the expanded configuration, an outer diameter (124) of the outer strut assembly (122) is greater than an average inner diameter of a human superior vena cava.

Clause 23. The intravascular blood pump (100) of any one of clauses 17-21,
wherein the filter section (120) further comprises a downstream anchor (132) coupled to the central body (136) upstream of the pump section (110),
wherein the filter section (120) further comprises an upstream anchor (130) coupled to the central body (136) upstream of the downstream anchor (132),
wherein an upstream end of the outer strut assembly (122) is coupled to the central body (136) by the upstream anchor (130), and
wherein a downstream end of the outer strut assembly (122) is coupled to the central body (136) by the downstream anchor (132).

Clause 24. The intravascular blood pump of clause 23, wherein one anchor of the upstream anchor (130) and the downstream anchor (132) is configured to slide along the longitudinal axis (LA), and the other anchor of the upstream anchor (130) and the downstream anchor (132) is affixed to the central body (136) and stationary with respect to the longitudinal axis (LA).

Clause 25. The intravascular blood pump (100) of clause 23 or 24,
wherein the filter section (12) further comprises a central anchor (134) couple to the central body (136) between the downstream anchor (132) and the upstream anchor (130),
wherein an upstream end of the inner strut assembly (126) is coupled to the central body (136) by the upstream anchor (130), and
wherein a downstream end of the inner strut assembly (126) is coupled to the central body (136) by the central anchor (134).

Clause 26. The intravascular blood pump (100) of clause 25, wherein the expandable structure is configured to direct blood flow toward the central anchor (134) in the expanded configuration.

Clause 27. The intravascular blood pump (100) of any one of clauses 1-26, wherein expandable structure (121) comprises a delivery configuration configured to be delivered through vasculature, wherein at least a portion of the expandable structure (121) spirals about the central body (136) in the delivery configuration.

Clause 28. The intravascular blood pump (100) of clause 10, wherein the removal configuration is distinct from the delivery configuration.

Clause 29. The intravascular blood pump (100) of any one of clauses 1-28, wherein, in the delivery configuration, the expandable structure (121) comprises a plurality of struts each respectively comprising a first strut portion which spirals clockwise about a first portion the central body (136) and a second strut portion which spirals counterclockwise about a second portion of the central body (136).

Clause 30. The intravascular blood pump (200) of any one of clauses 1-15,
wherein the filter section (220) comprises an anchor (132),
wherein the expandable structure comprises a first plurality of strut ends (234) affixed to the central body (136) by the anchor (132),
wherein the expandable structure extends upstream of the anchor and comprises a second plurality of strut ends (222) upstream of the anchor (132).

Clause 31. The intravascular blood pump (200) of clause 30, wherein the expandable structure comprises a plurality of struts arranged in a double wishbone configuration such that the expandable structure comprises:
a plurality of upstream strut extensions (224) extending downstream respectively from each of the second plurality of strut ends (222);
a plurality of downstream strut extensions (232) extending upstream respectively from each of the first plurality of strut ends (234); and
a plurality of wishbone struts (228) joining the plurality of upstream strut extensions (224) to the plurality of downstream strut extensions (232) such that the plurality of wishbone struts (228) extend from the plurality of upstream strut extensions at upstream wishbone splits (226) and the plurality of wishbone struts (228) extend from the plurality of downstream strut extensions at downstream wishbone splits (230).

Clause 32. An intravascular blood pump (200), comprising:
a pump section (110) comprising a blood flow inlet (112), a blood flow outlet (114), and an impeller (116) configured to cause blood to flow into the blood flow inlet (112) and towards the blood flow outlet (114); and
a filter section (220) disposed upstream of the pump section (110), the filter section (120, 220) comprising a central body (136) extending along a longitudinal axis (LA) and an expandable structure (221) disposed over the central body (136), the expandable structure (121, 221) being configured to radially expand away from the central body (136) in an expanded configuration,
wherein the expandable structure (221) comprises a plurality of struts arranged in a double wishbone configuration such that the expandable structure comprises:
a plurality of downstream strut extensions (232) extending upstream respectively from each of a first plurality of strut ends (234);
a plurality of upstream strut extensions (224) extending downstream respectively from each of a second plurality of strut ends (222); and
a plurality of wishbone struts (228) joining the plurality of upstream strut extensions (224) to the plurality of downstream strut extensions (232) such that the plurality of wishbone struts (228) extend downstream from the plurality of upstream strut extensions at upstream wishbone splits (226) and the plurality of wishbone struts (228) extend upstream from the plurality of downstream strut extensions at downstream wishbone splits (230).

Clause 33. The intravascular blood pump (200) of clause 32, wherein the expandable structure (221) lacks axial struts.

Clause 34. The intravascular blood pump (200) of clause 32 or 33, wherein every strut of the expandable structure (221) is non-orthogonal to the longitudinal axis (LA).

Clause 35. The intravascular blood pump (200) of any one of clauses 32-34, wherein the filter section (220) is disposed in a proximal direction in relation to the pump section (110).

Clause 36. The intravascular blood pump (200) of any one of clauses 32-35,
wherein the intravascular blood pump (200) is configured to be positioned such that the filter section (220) is disposed in a superior vena cava (SVC) and the blood flow outlet (114) is disposed in a pulmonary artery (PA), and
wherein the expandable structure (221) is configured to abut walls of the superior vena cava (SVC) in the expanded configuration.

Clause 37. The intravascular blood pump (200) of any one of clauses 32-36, wherein the expandable structure (221) comprises a memory shape material formed into a predetermined shape such that the expanded configuration is based at least in part on the predetermined shape.

Clause 38. The intravascular blood pump (200) of any one of clauses 32-37, wherein, in the expanded configuration, an outer diameter (236) of the expandable structure (221) is greater than an average inner diameter of a human superior vena cava.

Clause 39. A system (101) comprising:
an intravascular blood pump (100, 200) of any one of clauses 1-37;
an inner sheath (160) disposed over the expandable structure (121, 221) and constraining the expandable structure (121, 122) in a delivery configuration, wherein the sheath (160) is configured to be moved to uncover the expandable structure (121), and wherein the expandable structure (121) is configured to resiliently expand to the expanded configuration when unconstrained by the sheath (160); and
an outer sheath (164), wherein the inner sheath (160) is configured to be delivered through the outer sheath (164).

Clause 40. The system (101) of clause 39, wherein the inner sheath (160) comprises a 15 French outer diameter and at least an 11 French inner diameter.

Clause 41. The system (101) of clause 39 or 40, wherein the inner sheath (160) is configured to only partially collapse the expandable structure (121) from the expanded configuration.

Clause 42. The system (101) of any one of clauses 39-41, wherein the inner sheath (160) is configured to collapse the expandable structure (121) from the expanded configuration to a diameter less than an inner diameter of the inner sheath (160), and wherein the expandable structure (121) is configured to further collapse for removal through the outer sheath (164) upon encountering a distal end of the outer sheath (164).

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but, in any order, as long as the steps allow the embodiments to function for their intended purposes. Therefore, to the extent there are variations of the disclosure, which are within the spirit of the disclosure or equivalent to the disclosures found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. An intravascular blood pump comprising:
a pump section comprising a blood flow inlet, a blood flow outlet, and an impeller configured to cause blood to flow into the blood flow inlet and towards the blood flow outlet; and
a filter section disposed upstream of the blood flow inlet, the filter section comprising a central body extending along a longitudinal axis and an expandable structure disposed over the central body, the expandable structure being configured to radially expand away from the central body in an expanded configuration and direct blood flow toward the central body in the expanded configuration.

2. The intravascular blood pump of claim 1,
wherein the intravascular blood pump is configured to be positioned such that the filter section is disposed in a superior vena cava and the blood flow outlet is disposed in a pulmonary artery, and
wherein the expandable structure is configured to abut walls of the superior vena cava in the expanded configuration.

3. The intravascular blood pump of claim 2, wherein the expandable structure is configured to capture thrombus.

4. The intravascular blood pump of claim 3, wherein the expandable structure is configured to direct thrombus toward the central body and retain the thrombus within the filter section.

5. The intravascular blood pump of claim 4, wherein the filter section is configured to inhibit thrombus large enough to impede the impeller from encountering the blood flow inlet due to blood flow caused by the impeller.

6. The intravascular blood pump of claim 5, wherein the filter section is configured to allow small thrombus to encounter the blood flow inlet such that the small thrombus is of a size capable of entering the blood flow inlet and exiting the blood flow outlet.

7. The intravascular blood pump of claim 1, wherein the expandable structure comprises a plurality of struts shaped to direct blood flow toward the central body in the expanded configuration.

8. The intravascular blood pump of claim 7, wherein every strut of the expandable structure comprises two ends such that one of the two ends is upstream of the other of the two ends.

9. The intravascular blood pump of claim 8, wherein the expandable structure comprises a delivery configuration in which the expandable structure is radially constrained by a sheath.

10. The intravascular blood pump of claim 9, wherein the expandable structure comprises a delivery configuration and a removal configuration such that the expandable structure is configured to resiliently expand from the delivery configuration to the expanded configuration upon removal from a sheath and be collapsed by the sheath to the removal configuration.

11. The intravascular blood pump of claim 1, wherein the filter section is disposed in a proximal direction in relation to the pump section.

12. The intravascular blood pump of claim 1, wherein an upstream direction and a downstream direction are defined by a blood flow direction across the filter section and through the pump section.

13. The intravascular blood pump of claim 1, wherein the expandable structure comprises a memory shape material formed into a predetermined shape such that the expanded configuration is based at least in part on the predetermined shape.

14. The intravascular blood pump of claim 1, further comprising:
an elongated catheter body configured to be manipulated to position the pump section and the filter section within a heart.

15. The intravascular blood pump of claim 14, wherein the pump section further comprises a motor disposed in a motor housing and configured to drive the impeller.

16. The intravascular blood pump of claim 15, wherein at least a portion of the plurality of struts spiral about the central body in the expanded configuration.

17. The intravascular blood pump of claim 16, wherein the expandable structure comprises an outer strut assembly and an inner strut assembly such that the outer strut assembly defines an inner volume in the expanded configuration and at least a majority of the inner strut assembly is disposed within the inner volume.

18. The intravascular blood pump of claim 17, wherein the outer strut assembly forms a lemon shape in the expanded configuration.

19. The intravascular blood pump of claim 17, wherein the inner strut assembly forms an onion shape in the expanded configuration.

20. The intravascular blood pump of claim 17, wherein at least a portion of each strut of the inner strut assembly spirals about the central body in the expanded configuration.
